(19) United States
(12) Patent Application Publication
(10) Pub. No.: US 2014/0249419 A1
(43) Pub. Date: Sep. 4, 2014

(54) METHOD FOR MANUFACTURING ULTRASOUND PROBE AND ULTRASOUND DIAGNOSTIC IMAGING APPARATUS

(71) Applicant: KONICA MINOLTA, INC., Tokyo (JP)

(72) Inventor: Kiyokazu MORITA, Tokyo (JP)

(73) Assignee: KONICA MINOLTA, INC., Tokyo (JP)

(21) Appl. No.: 14/194,166

(22) Filed: Feb. 28, 2014

(30) Foreign Application Priority Data
Mar. 1, 2013 (JP) 2013-040215

(51) Int. Cl. A61B 8/00

(52) U.S. Cl. CPC A61B 8/4444
USPC 600/459; 156/60

(57) ABSTRACT

A method for manufacturing an ultrasound probe which includes a piezoelectric element for transmitting and receiving ultrasound waves and an acoustic matching layer provided by lamination of two or more matching materials on a front surface of the piezoelectric element, the method including forming at least one of the two or more matching materials from a thermosetting resin and a filler for adjusting an acoustic impedance; and bonding the two or more matching materials with an adhesive having a viscosity of 10 Pa·s or less at 25° C.

### BACKGROUND OF THE INVENTION

1. Field of the Invention

The present invention relates to a method for manufacturing an ultrasound probe and an ultrasound diagnostic imaging apparatus.

2. Description of Related Art

An ultrasound probe used in a typical ultrasound diagnostic imaging apparatus includes, for example, an acoustic lens, an acoustic matching layer, a piezoelectric element, and a backing layer. These components are bonded with, for example, an epoxy resin adhesive or a silicone resin adhesive into an integrated unit. In this structure, the acoustic matching layer preferably has variable acoustic impedance along a sound axis. More specifically, the acoustic matching layer preferably has an acoustic impedance closer to the acoustic impedance of the piezoelectric element at a position closer to the piezoelectric element, and a lower acoustic impedance, i.e., an acoustic impedance closer to the acoustic impedance of a subject such as a living body at a position closer to the living body as the subject, i.e., further from the piezoelectric element. An ultrasound probe having such an acoustic matching layer can effectively transmit and receive ultrasound waves to and from a subject.

In order to widen the band of frequency characteristics of an ultrasound probe, it is known that such an acoustic matching layer is formed by laminating three or more layers of matching materials. In some of such acoustic matching layers, an acoustic matching layer close to a piezoelectric elements which is composed of a metal matching material such as a magnesium alloy is disclosed in Japanese Patent Application Laid Open Publication No. 2008-244859, for example.

Acoustic matching layers which are composed of a polymer alloy of polyether-modified resin having acoustic impedance of 1.6 to 2.5 MRayls at a portion close to an acoustic lens are disclosed in Japanese Patent Application Laid Open Publication No. 2007-189342, for example.

### SUMMARY OF THE INVENTION

However, in a case where a matching material composed of a single material described in Japanese Patent Application Laid Open Publication No. 2008-244859 and Japanese Patent Application Laid Open Publication No. 2007-189342 is used, the acoustic impedance, which is inherent in the substance, can barely be adjusted to any value, and adjustment of the acoustic impedance is difficult. For example, when using a piezoelectric material such as a composite piezoelectric material composed of a piezoelectric material and a resin or a single-crystal piezoelectric material, which has an acoustic impedance different from that of ceramic material such as lead zirconate titanate (PZT), a matching material having appropriate acoustic impedance cannot be readily found.

Thus, a possible approach to adjust the acoustic impedance of the matching material is filling a resin with a filler to generate a matching material and adjusting the filling amount of the filler.

Lamination of matching materials to form an acoustic matching layer having multiple layers includes a process for bonding the matching materials with an adhesive. Since an adhesive used in conventional techniques has high viscosity, the adhesive layer must be thinned after the lamination of the matching materials to bond them with the adhesive in the bonding process. This requires press of the laminated matching materials under a predetermined pressure to squeeze the excess adhesive out of the laminate. If a matching material including a resin filled with filler is used in this process, the material may be damaged due to a shearing stress caused by the pressure. If the pressure for the bonding is reduced so as not to damage such a matching material, the adhesive layer has a large thickness and serves as an acoustic impedance inversion layer. This cannot sufficiently widen the band of the frequency characteristics.

The present invention has been made in consideration of the above matters, and an object of the present invention is to provide a method for manufacturing an ultrasound probe and an ultrasound diagnostic imaging apparatus that can prevent matching materials from being damaged in a bonding process for the matching materials.

In order to achieve at least one of the above objects, according to one aspect of the present invention, there is provided a method for manufacturing an ultrasound probe which includes a piezoelectric element for transmitting and receiving ultrasound waves and an acoustic matching layer provided by lamination of two or more matching materials on a front surface of the piezoelectric element, the method including forming at least one of the two or more matching materials from a thermosetting resin and a filler for adjusting an acoustic impedance; and bonding the two or more matching materials with an adhesive having a viscosity of 10 Pa·s or less at 25° C.

Preferably, in the above method for manufacturing the ultrasound probe, the adhesive has a glass transition temperature of 50° C. or more after curing.

Preferably, in the above method for manufacturing the ultrasound probe, a matching material which is a foremost layer in the acoustic matching layer contains silicone resin particles.

Preferably, in the above method for manufacturing the ultrasound probe, the bonding of the two or more matching materials with the adhesive includes a first step of holding the two or more matching materials at a predetermined temperature for a predetermined period until a thickness of an adhesive layer reaches a predetermined thickness from a start of the bonding and a second step of subsequently holding the two or more matching materials at a higher temperature.

Preferably, in the above method for manufacturing the ultrasound probe, the two or more matching materials are held at 15° C. or more and 30° C. or less for 2 to 6 hours in the first step and at 60° C. or less for 1 to 4 hours in the second step.

In order to achieve at least one of the above objects, according to another aspect of the present invention, there is provided an ultrasound diagnostic imaging apparatus including an ultrasound probe manufactured by the above method for manufacturing the ultrasound probe, the ultrasound probe outputting transmission ultrasound toward a subject in response to a driving signal and receiving reflected ultrasound from the subject to output a received signal; and an image processing unit for generating ultrasound image data for displaying an ultrasound image based on the received signal outputted from the ultrasound probe.

### BRIEF DESCRIPTION OF THE DRAWINGS

The above and other objects, advantages and features of the present invention will become more fully understood from the detailed description given hereinbelow and the appended drawings which are given by way of illustration only, and thus are not intended as a definition of the limits of the present invention, and wherein:

FIG. 1 illustrates an outline view of an ultrasound diagnostic imaging apparatus;

FIG. 2 is a block diagram illustrating the outline configuration of the ultrasound diagnostic imaging apparatus;

FIG. 3 is a sectional view illustrating the outline configuration of an ultrasound probe;

FIG. 4 is a sectional view illustrating the outline configuration of an ultrasound probe according to another embodiment; and

FIG. 5 is a sectional view illustrating the outline configuration of a piezoelectric element.

### DETAILED DESCRIPTION OF THE PREFERRED EMBODIMENT

Hereinafter, an ultrasound diagnostic imaging apparatus according to an embodiment of the present invention will now be described with reference to the accompanying drawings. The scope of the present invention should however not be limited to examples illustrated in these drawings. In the following description, elements having the same functions and configurations are designated with the same reference numerals, without redundant description.

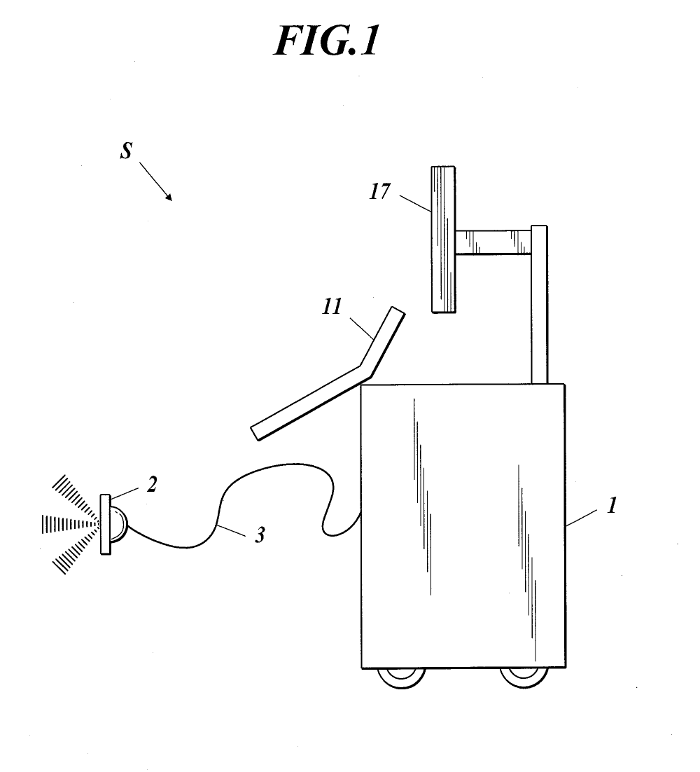

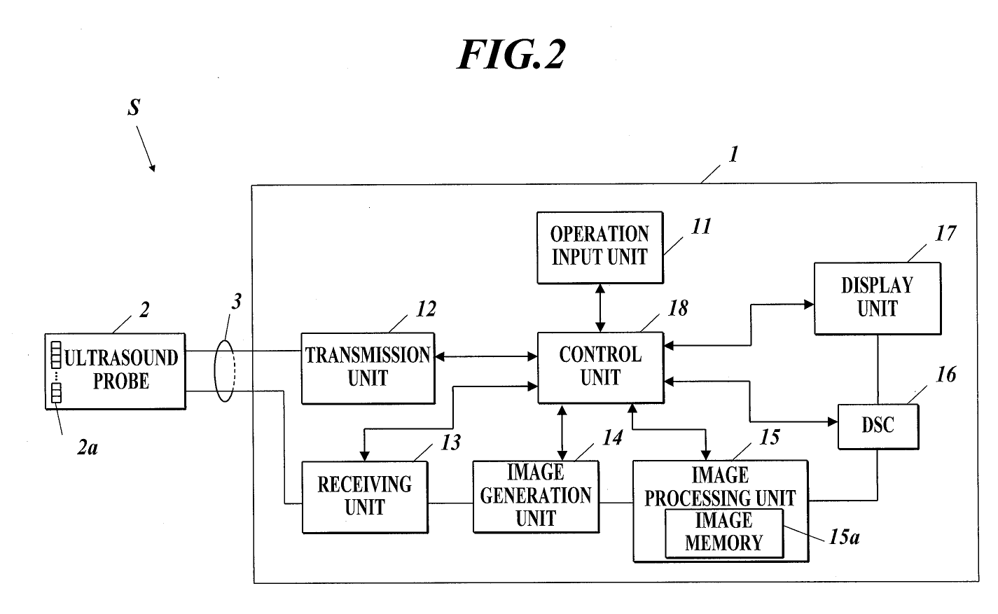

The ultrasound diagnostic imaging apparatus S according to the embodiment includes an ultrasound diagnostic imaging apparatus main body 1 and an ultrasound probe 2 as illustrated in FIGS. 1 and 2. The ultrasound probe 2 transmits ultrasound waves (transmission ultrasound) toward a subject, such as a living body (not illustrated) and receives ultrasound waves reflected (reflected ultrasound: echo) from this subject. The ultrasound diagnostic imaging apparatus main body 1 is connected to the ultrasound probe 2 through a cable 3, transmits driving signals as electrical signals to the ultrasound probe 2 to cause the ultrasound probe 2 to transmit transmission ultrasound toward the subject, and generates an ultrasound image of the internal state of the subject on the basis of received signals as electrical signals generated in the ultrasound probe 2 in response to reflected ultrasound from the interior of the subject received at the ultrasound probe 2.

The ultrasound probe 2 includes multiple vibrators 2a composed of piezoelectric elements (refer to FIG. 2), the multiple vibrators 2a being arranged, for example, in a one-dimensional array in the orientation direction. The embodiment involves the ultrasound probe 2 including, for example, 192 vibrators 2a. The vibrators 2a may be arranged in a two-dimensional array. The number of the vibrators 2a may be set to any number. The ultrasound probe 2 used in the embodiment is an electronic-scanning linear probe. Alternatively, the probe may be of an electronic or mechanical scanning type and any type of linear scanning, sector scanning, and convex scanning.

As illustrated in FIG. 2, the ultrasound diagnostic imaging apparatus main body 1 includes, for example, an operation input unit 11, a transmission unit 12, a receiving unit 13, an image generation unit 14, an image processing unit 15, a digital scan converter (DSC) 16, a display unit 17, and a control unit 18.

The operation input unit 11 includes, for example, various switches and buttons, a trackball, a mouse, a keyboard for inputting a command instructing the start of a diagnostic and data, such as personal information on the subject, and outputs operation signals to the control unit 18.

The transmission unit 12 is a circuit supplying driving signals as electrical signals to the ultrasound probe 2 through the cable 3 and causes the ultrasound probe 2 to generate transmission ultrasound under control of the control unit 18. The transmission unit 12 includes, for example, a clock generator circuit, a delay circuit, and a pulse generator circuit. The clock generator circuit is a circuit generating clock signals for determining the transmission timing and transmission frequency of driving signals. The delay circuit sets the delay time for the corresponding path for the each vibrator and delays the transmission of driving signals with the set delay time to focus a transmission beam (transmission beam forming) consisting of the transmission ultrasound. The pulse generator circuit generates pulse signals as driving signals in a predetermined cycle. The transmission unit 12 arranged as described above drives, for example, several (for example, 64) successive vibrators among the vibrators (for example, 192 vibrators) arranged in the ultrasound probe 2 and generates transmission ultrasound. For acquisition of an ultrasound image in a B mode described below, the transmission unit 12 performs scanning by shifting the vibrator to be driven in the orientation direction every time generating transmission ultrasound. In the embodiment, the transmission unit 12 can also cause the ultrasound probe 2 to generate transmission ultrasound including pulse waves for displaying an ultrasound image generated by a pulse-Doppler system.

The receiving unit 13 is a circuit receiving received signals as electrical signals from the ultrasound probe 2 through the cable 3 under the control of the control unit 18. The receiving unit 13 includes, for example, an amplifier, an A/D conversion circuit, and a phasing addition circuit. The amplifier is a circuit for amplifying received signals with a predetermined gain for the corresponding path for each vibrator 2a. The A/D conversion circuit performs analog-to-digital (A/D) conversion of the amplified received signal. The phasing addition circuit applies a delay time to the A/D converted received signals for the corresponding path for each vibrator 2a to set time phase and adds (phasing addition) these signals to generate sound ray data.

The image generation unit 14 applies an envelope detection process or logarithmic amplification to the sound ray data from the receiving unit 13 and generates B mode image data through brightness conversion with, for example, gain adjustment. In other words, the B mode image data includes brightness representing the intensity of received signal. The B mode image data generated in the image generation unit 14 is transmitted to the image processing unit 15.

The image processing unit 15 includes an image memory unit 15a composed of a semiconductor memory, such as a dynamic random access memory (DRAM). The image processing unit 15 stores the B mode image data outputted from the image generation unit 14 on a frame basis in the image memory unit 15a. The image data on a frame basis may be referred to as ultrasound image data or frame image data. The frame image data stored in the image memory unit 15a is transmitted to the DSC 16 under the control of the control unit 18.

The DSC 16 converts the ultrasound diagnostic image data received from the image processing unit 15 into image signals based on a scanning scheme of television signals and outputs the signals on the display unit 17.

The display unit 17 may be a liquid crystal display (LCD), a cathode-ray tube (CRT) display, an organic electronic luminescence (EL) display, an inorganic EL display, or a plasma display. The display unit 17 displays an ultrasound diagnostic image on a display screen in response to the image signals outputted from the DSC 16. Alternatively, the display may be replaced with a printing apparatus such as a printer.

The control unit 18 includes, for example, a central processing unit (CPU), a read only memory (ROM), and a random access memory (RAM) and reads out various programs such as system programs stored in the ROM to expand the programs in the RAM, and exerts centralized control on individual units of the ultrasound diagnostic imaging apparatus S according to the expanded programs.

The ROM is composed of a nonvolatile memory, such as, a semiconductor and stores, for example, various pieces of data, and system programs and various processing programs performable on the system programs adapted for the ultrasound diagnostic imaging apparatus S. These programs are stored in a form of computer-readable program codes. The CPU sequentially operates according to the program codes.

The RAM has a work area temporarily storing various programs performed by the CPU and data in association with these programs.

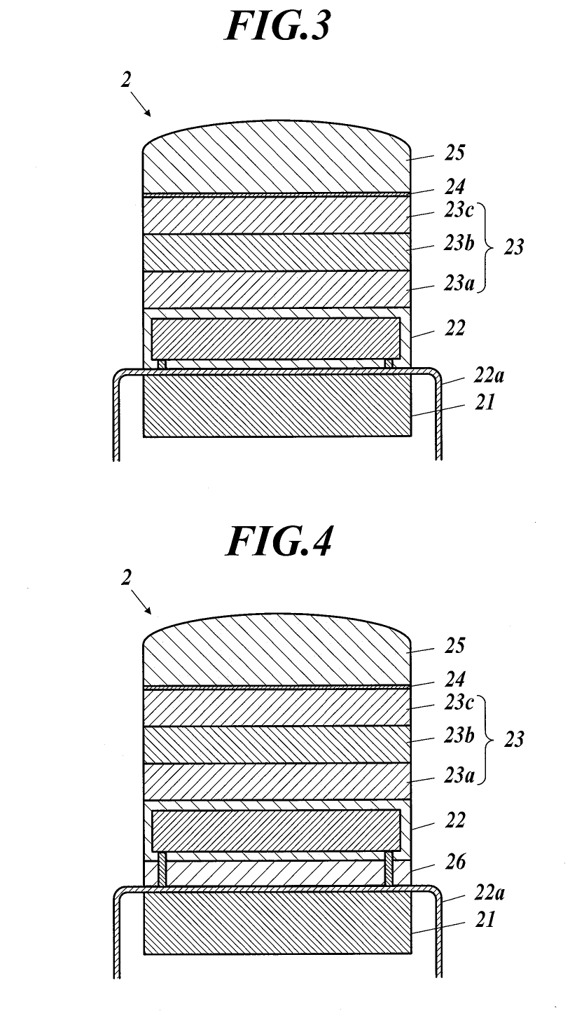

The ultrasound probe 2 according to the embodiment will now be described with reference to FIG. 3.

From the lower part of FIG. 3, the ultrasound probe 2 includes a backing layer 21, a piezoelectric layer 22 stacked on a flexible printed circuit board (FPC) 22a provided on the backing layer 21, an acoustic matching layer 23 stacked on (the front surface of) the piezoelectric layer 22, a protection layer 24 stacked on the acoustic matching layer 23, and an acoustic lens 25 stacked on the protection layer 24.

The backing layer 21 is an ultrasound absorber that supports the piezoelectric layer 22 and can absorb unnecessary ultrasound waves. More specifically, the backing layer 21 is mounted on the opposite surface of the piezoelectric layer 22 remote from the side of transmission and reception of sound waves to the subject and absorbs ultrasound waves generated from the opposite side to the subject.

Examples of the backing material for the backing layer 21 include natural rubber, ferrite rubber, and epoxy resin; rubber composites and epoxy resin composites which are compressed composites of these materials with powder such as tungsten oxide, titanium oxide, and ferrite; and thermoplastic resins, such as vinyl chloride, polyvinyl butyral (PVB), ABS resin, polyurethane (PUR), polyvinyl alcohol (PVAL), polyethylene (PE), polypropylene (PP), polyacetal (POM), polyethylene terephthalate (PETP), fluorinated resins (PTFE) polyethylene glycol, copolymers of polyethylene terephthalate and polyethylene glycol.

A preferred backing material includes a rubber composite and/or an epoxy resin composite, and its shape may be suitably selected depending on the shape of the piezoelectric layer 22 or a probe head including this layer.

The piezoelectric layer 22 has electrodes and a piezoelectric material and is an element (piezoelectric element) that can convert electrical signals into mechanical vibration and mechanical vibration into electrical signals and can transmit and receive ultrasound waves.

The piezoelectric material contains a piezoelectric substance that can convert electrical signals into mechanical vibrations and mechanical vibrations into electrical signals. The piezoelectric substance may be, for example, piezoelectric ceramics, such as lead zirconate titanate (PZT) ceramics, lead titanate, and lead metaniobate; a piezoelectric single crystal consisting of solid-solution single crystals, such as lithium niobate, lead zinc niobate and lead titanate, and lead magnesium niobate and lead titanate; quartz; Rochelle salt; and organic polymer piezoelectric materials; such as PVDF copolymers, e.g., polyvinylidene-fluoride-trifluorochloroethylene (poly(VDF-TrFE)), which is a copolymer of polyvinylidene fluoride (PVDF) or VDF with trifluorochloroethylene (TrFE); polyvinylidene cyanide (PVDCN), which is a polymer of vinylidene cyanide (VDCN) or a vinylidene cyanide copolymer; odd nylons, such as nylon 9 and nylon 11; aromatic nylons; alicyclic nylons; polylactic acid; polyhydroxy carboxylic acids, such as polyhydroxybutyrate; cellulose derivatives; and polyurea. Alternatively, a composite piezoelectric substance may be available that is formed by alternately disposing layers of a piezoelectric material described above and polymer layers composed of, for example, an epoxy resin or a silicone resin in a one-dimensional array.

The thickness of the piezoelectric material ranges from about 100 to 500 μm. The piezoelectric material has electrodes on both sides and serves as the vibrator 2a.

Examples of the material used for the electrodes attached to the piezoelectric material include gold (Au), platinum (Pt), silver (Ag), palladium (Pd), copper (Cu), aluminum (Al), nickel (Ni), and tin (Sn).

The electrodes are provided to the piezoelectric material, for example, through formation of underlaying metal, such as titanium (Ti) or chromium (Cr), into a thickness of 0.02 to 1.0 μm through spattering and then formation of a metal material mainly composed of the metallic elements described above, alloys of these metals, and a partial insulating material, if needed, into a thickness of 1 to 10 μm by spattering or any other appropriate means.

Instead of spattering, the electrodes may also be formed by applying conductive paste that is the mixture of fine metal powder and low-melting glass through screen printing, dipping, or thermal spraying. The electrode is provided on the entire or partial surface of the piezoelectric substance on the piezoelectric material depending on the shape of the ultrasound probe 2.

The piezoelectric layer 22 and the backing layer 21 are preferably bonded with the adhesive layer. The adhesive for the adhesive layer may be an epoxy adhesive.

The piezoelectric layer 22 has the electrodes in contact with the FPC 22a that is electrically connected to the cable 3. In other words, driving signals outputted from the ultrasound diagnostic imaging apparatus main body 1 are inputted to the piezoelectric layer 22 through the FPC 22a, and received signals generated in the piezoelectric layer 22 are outputted to the ultrasound diagnostic imaging apparatus main body 1.

The acoustic matching layer 23 matches the acoustic impedance between the piezoelectric layer 22 and the subject and reduces the reflection on the boundary surface. The acoustic matching layer 23 is mounted adjacent to the subject, i.e., on the side of the piezoelectric layer 22 of transmission and reception of ultrasound waves.

The acoustic matching layer 23 is formed by laminating a bottommost matching material 23a, a middle matching material 23b, and a topmost (foremost) matching material 23c. The acoustic matching layer 23 may include at least two laminated sublayers, preferably three or more sublayers. The thickness of the acoustic matching layer 23 is preferably determined to be λ/4 where the wavelength of ultrasound waves is λ. An inappropriate thickness of the acoustic matching layer 23 may cause multiple unnecessary spuriouses at frequency points different from an original resonance frequency to significantly vary basic acoustic characteristics. This may lead to an increase in reverberation time, or a decrease in the sensitivity or S/N due to the waveform distortion of reflective echoes. The thickness of such an acoustic matching layer usually ranges from about 20 to 500 μm.

The acoustic matching layer 23 has an acoustic impedance substantially intermediate between the piezoelectric layer 22 and the subject. The acoustic matching layer 23 has an acoustic impedance gradually decreasing from the bottommost matching material 23a to the topmost matching material 23c, and the topmost matching material 23c has a higher acoustic impedance than that of the protection layer 24 described below. Additionally, the acoustic impedance of the topmost matching material 23c preferably ranges from 1.3 to 2.5 MRayls for matching with the acoustic impedance of the acoustic lens 25 described below.

Examples of the material used for the acoustic matching layer 23 include aluminum, aluminum alloys (for example, an AL-Mg alloy), magnesium alloys, Macor glass, glass, fused quartz, copper graphite, polyethylene (PE), polypropylene (PP), polycarbonates (PCs), ABC resins, ABS resins, AAS resins, AES resins, nylons (PA6, PA6-6), polyphenylene oxide (PPO), polyphenylene sulfide (PPS, may contain glass fiber) and polyphenylene ethers (PPEs), polyether ether ketones (PEEKs), polyamide imides (PAIs), polyethylene terephthalate (PETP), epoxy resins, and urethane resins. Preferred materials are thermosetting resins, such as epoxy resins, that contain fillers such as zinc oxide, titanium oxide, silica, alumina, red oxide, ferrite, tungsten oxide, ytterbium oxide, barium sulfate, tungsten, and/or molybdenum. The addition of the fillers to the thermosetting resins and variation in the specific gravity of the filler can appropriately adjust the acoustic impedance of the matching material of each sublayer included in the acoustic matching layer 23.

In a preferred embodiment, the topmost matching material 23c contains at least silicone resin particles. Alternatively, the topmost matching material 23c may also contain any other material instead of silicone particles to decrease the acoustic impedance.

Examples of silicone particles used in the embodiment include particulate crosslinked polydimethylsiloxane and crosslinked polydimethylsiloxane of which the surface may be coated with a silicon resin. Examples of such particles composed of crosslinked polydimethylsiloxane include EP5500, EP2600, EP2601, EP-2720, and E-606 (produced by Dow Corning Toray Co., Ltd.); KMP-400, KMP-591, KMP-597, KMP-594, KMP-598, X-52-875, KMP-590, KMP-701, X-52-854, and X-2-1621 (produced by Shin-Etsu Chemical Co., Ltd.); Tospearls 120, 130, 145, 2000B, 1110, and 240 (produced by Momentive Performance Materials Inc.). Examples of such particles having surfaces coated with a silicon resin include KMP-600, KMP-601, KMP-602, KMP-605, X-52-7030, KSP-100, KSP-101, KSP-102, KSP-105, and KSP-300 (produced by Shin-Etsu Chemical Co., Ltd.).

In the embodiment, a substance formed by coating the surface of crosslinked polydimethylsiloxane particles with a silicon resin is preferred from the viewpoint of the compatibility of a resin component with silicone particles. Such particles are, for example, disclosed in Japanese Patent Application Laid Open Publication No. 7-196815. Silicone particles contained in the matching material preferably have a sufficiently smaller average particle diameter than the thickness of the sublayer. If the particle diameter is not sufficiently smaller than the layer, the matching material contains portions of the silicone particles and portions of the epoxy resin as the base material, resulting in significant local variations in the physical properties. This leads to fluctuated characteristics of the completed ultrasound probes, or causes the silicone particles to fall out during the manufacture of the matching material and causes deficiencies (cavities) in the matching material. More specifically, the embodiment may preferably use particles having a diameter of 10 μm or less.

The silicone particles, which decrease the specific gravity and the sound velocity in correlation to their content, can reduce the acoustic impedance. In the embodiment, the silicone particles are added in an amount of up to 200 parts by weight preferably at most 120 parts by weight relative to 100 parts by weight of epoxy resin. If the content exceeds this upper limit, the composition may have high viscosity to significantly decrease the workability.

As described above, the acoustic matching layer 23 is formed by laminating and bonding three or more matching material sublayers with an adhesive described below and pressing the sublayers until each adhesive layer has a thickness less than 1 μm. In this process for bonding the matching materials, a first step is pressure-holding at 15° C. or more and 30° C. or less for 2 to 6 hours, and then a second step is pressure-holding at 60° C. or less for 1 to 4 hours. The first step enables the adhesive layer for bonding the matching materials 23a to 23c to have a smaller thickness than a predetermined value and the second step can enhance the adhesive strength. These steps can also provide a wide applicable range of the adhesive.

The embodiment may use an epoxy resin adhesive having low viscosity. This can prevent cracking due to shear stress in the bottommost matching material 23a, in particular, having large specific gravity for increasing the acoustic impedance during the compression of the laminated matching materials. In this process, the adhesive preferably has a glass transition temperature (Tg) of 50° C. or more. This can prevent separation of the matching materials due to deterioration of the adhesive caused by heat generated during the manufacture of elements by dicing. Alternatively, the adhesive may have a glass transition temperature (Tg) of less than 50° C.

The embodiment may use any known epoxy resin as the adhesive without limitation. Examples of such epoxy resins include glycidyl ether epoxy resins, glycidyl ester epoxy resins, glycidylamine epoxy resins, and oxidized epoxy resins.

Examples of the glycidyl ether epoxy resins include bisphenol A epoxy resins, bisphenol F epoxy resins, novolac epoxy resins, and alcohol epoxy resins. Examples of the glycidyl ester epoxy resins include hydrophthalic acid epoxy resins and dimer acid epoxy resins. Examples of the glycidylamine epoxy resins include aromatic amine epoxy resins and aminophenol epoxy resins. Examples of the oxidized epoxy resin include alicyclic epoxy resins. Further examples include epoxy resins having naphthalene skeletons, novolac epoxy resins having phenol skeletons and biphenyl skeletons (biphenyl novolak epoxy resins), phosphorus-modified epoxy resins, and liquid crystal epoxy resins. The embodiment may use a single epoxy resin component or a mixture of two or more epoxy resin components like blended resins. The epoxy resin composition in this embodiment may contain, for example, thermoplastic resins soluble in the epoxy resin; organic particles, such as, rubber particles and thermoplastic resin particles; and/or inorganic particles, in order to control the viscoelasticity and to improve the dynamic properties, such as adhesiveness.

The epoxy resin to be used may have any epoxy equivalent which preferably ranges from 100 to 1000000 (g/eq), more preferably from 100 to 10000 (g/eq) from the viewpoint of providing more excellent adhesiveness of a metal film.

The viscosity (25° C.) of the epoxy resin preferably ranges from 0.01 to 10 Pa·s, more preferably from 0.01 to 3 Pa·s from the viewpoint of advantages in the thickness, handling, and adhesiveness of the adhesive layers. The viscosity of the epoxy resin is measured at 25° C. with a typical viscometer (for example, type E viscometer (RE-80L) produced by Toki Sangyo Co., Ltd). Among such liquid epoxy resins, preferred are glycidyl ethers based on bisphenol A or F, which has low viscosity.

A curing agent for an epoxy resin used in the embodiment is heated at 50 to 200° C. to cause a crosslinking reaction with the epoxy groups in the epoxy resin and cures the epoxy resin composition.

Such curing agents may be typical curing agents that have been used for epoxy resins.

Examples of the curing agents include dicyandiamide; 4,4′-diaminodiphenyl sulfone; imidazole derivatives such as 2-n-heptadecyl imidazole; isophthalic acid dihydrazide; N,N-dialkyl urea derivatives; N,N-dialkyl thiourea derivatives; acid anhydrides such as tetrahydrophthalic anhydride; polyamines such as isophoronediamine, m-phenylenediamine; ethylenediamine, hexamethylenediamine, m-xylenediamine, diethylenetriamine, triethylenetetramine, and tetraethylenepentamine; aminoalkyl ring compounds such as bis(aminomethyl)cyclohexane, N-aminoethylpiperazine, tris(dimethylaminomethyl)phenol, and 3,9-bis(3-aminopropyl)-2,4,8,10-tetraoxaspiro(5,5)undecane; melamine; boron fluoride complex compounds; and polyamideamines being addition compounds of various dimer acids with diamines.

These curing agents for epoxy resins may be used alone or in combination.

Examples of effect auxiliary agents for these epoxy resin components include 1,8-diaza-bicyclo(5,4,0)undecene-7; triethylenediamine; tertiary amine compounds such as benzyldimethylamine; imidazoles such as 2-methylimidazole, 2-ethyl-4-methylimidazole, 2-phenylimidazole, and 2-phenyl-4-methylimidazole; and organic phosphine compounds such as triphenylphosphine and tributylphosphine.

The proportion of the curing agent in the liquid epoxy resin composition usually ranges from 3 to 100 parts by weight relative to 100 parts by weight of epoxy resin. The ratio of the curing accelerator to the curing agent is equal to or less than about ⅕.

The protection layer 24 is composed of a gas barrier material that can prevent washing solution from intruding through the acoustic lens 25 into the components in the acoustic lens 25 and the adhesive layer between the components during washing before and after the measurement and that enables highly accurate measurement. The protection layer 24 is composed of a material having an acoustic impedance of 2.5 MRayls or less. In addition to the material having the acoustic impedance described above, any material having an acoustic impedance smaller than the acoustic impedance of the topmost layer 23c of an acoustic matching layer 23 and larger than the acoustic impedance of the acoustic lens 25 may also applied to the protection layer 24. The material of the protection layer 24 may be composed of a compound having a polyparaxylylene structure. The material of the protection layer 24 may also be composed of a polyurea resin.

The protection layer 24 according to the embodiment may have any thickness, which is preferably 0.5 to 5.0 μm, more preferably 2.0 to 4.0 μm.

The acoustic lens 25 focuses the ultrasound beam by refraction to improve the resolution. More specifically, the acoustic lens 25 is provided so as to contact the subject for the ultrasound probe 2 and facilitates the incidence of ultrasound waves generated in the piezoelectric layer 22 onto the subject. The acoustic lens 25 is a convex or concave lens depending on the internal sound velocity in the portion in contact with the subject, and focuses ultrasound waves incident on the subject in the thickness direction (elevation direction) orthogonal to an imaging cross section.

The acoustic lens 25 is composed of an elastic polymer material having an acoustic impedance substantially intermediate between that of the subject and that of the acoustic matching layer 23.

Examples of materials used for the acoustic lens 25 include known silicone rubbers; butadiene rubbers; polyurethane rubbers; homopolymers, such as epichlorohydrin rubbers; and copolymer rubbers, such as ethylene-propylene copolymer rubbers based on copolymerization of ethylene and propylene. Silicone rubbers and butadiene rubbers are preferred among these compounds.

Examples of silicone rubbers used in the embodiment include untreated silicone rubbers and fluorinated silicone rubbers. Untreated silicone rubbers are preferably used from the viewpoint of the properties of a lens material. Silicone rubbers have a molecular framework including Si—O bonds, are organopolysiloxanes having primary bonds between Si atoms and multiple organic groups, and usually contain methyl polysiloxane as a main component. At least 90% of all the organic groups are methyl groups. The methyl groups may be replaced with hydrogen atoms, phenyl groups, vinyl groups, or allyl groups. This silicone rubber can be prepared, for example, by kneading a curing agent (vulcanizing agent), such as benzoyl peroxide with organopolysiloxane having a high degree of polymerization, and then heating, and curing by vulcanizing the kneaded compound. Organic or inorganic fillers, such as silica and nylon powder; and vulcanizing auxiliary agents, such as sulfur and zinc oxide may be added if necessary.

Examples of butadiene rubbers used in the embodiment include butadiene homopolymers and copolymers of a major amount of butadiene and a minor amount of styrene or acrylonitrile. Butadiene rubbers are preferably used from the viewpoint of the properties of a lens material. The butadiene rubbers refer to synthetic rubbers formed by polymerization of butadiene, which has a conjugate double bond. Butadiene rubbers can be prepared through 1.4- or 1.2-polymerization of pure butadiene having a conjugate double bond. The butadiene rubbers can be prepared through vulcanization with, for example, sulfur.

The acoustic lens 25 in the present invention can be obtained by mixing silicon rubber with butadiene rubber and curing the mixed compound through vulcanization. For example, the acoustic lens can be prepared by mixing silicone rubber with butadiene rubber at an appropriate ratio through a kneading roll; adding a vulcanizing agent, such as benzoyl peroxide thereto; and achieving crosslinking (curing) through thermal vulcanization. In this process, zinc oxide is preferably added as a vulcanizing auxiliary agent. Zinc oxide can accelerate vulcanization and shorten the vulcanizing time while maintaining desirable lens properties. In addition, other additives may be added within the range where properties of a colorant and an acoustic lens are not impaired. A mixing ratio of silicon rubber to butadiene rubber is preferably 1:1 in the usual case in order to provide an acoustic impedance close to that of a human body, a smaller sound velocity than that of the human body, and small attenuation, and may be appropriately changed.

Silicone rubber is commercially available, and usable examples thereof include KE742U, KE752U, KE931U, KE941U, KE951U, KE961U, KE850U, KE555U, and KE575U produced by Shin-Etsu chemical Co., Ltd.; TSE221-3U, TE221-4U, TSE2233U, XE20-523-4U, TSE27-4U, TSE260-3U, and TSE-260-4U produced by Momentive Performance Materials Inc.; and SH35U, SH55UA, SH831U, SE6749U, and SE1120USE4704U produced by Dow Corning Toray Co., Ltd.

In the embodiment, a raw rubber material such as the silicon based rubber described above may be used as a base (main component), and inorganic fillers, such as silica, alumina, and titanium oxide; and organic resins, such as nylons can be blended, for example, for adjustment of the sound velocity or the density.

In the embodiment, the ultrasound probe 2 is used as described above. Alternatively, as shown in FIG. 4, a heavy backing layer 26 may be provided between the piezoelectric layer 22 and the backing layer 21. The heavy backing layer 26 is composed of a material having a larger acoustic impedance than that of the piezoelectric layer 22 and reflects ultrasound waves outputted in a direction opposite to the subject toward the piezoelectric layer 22. The heavy backing layer 26 can further improve the sensitivity to transmission and reception of ultrasound waves in the piezoelectric layer 22. The addition of the heavy backing layer 26 narrows the bandwidth. Instead, three or more sublayers in the acoustic matching layer 23 as illustrated in FIG. 4 can widen the bandwidth.

### Example 1

The present invention will be described in more detail with reference to examples, but should not be limited to these examples.

### <Production of Matching Materials>

To 25 parts by weight of silicon compound powder KMP600 (produced by Shin-Etsu Silicon) was added 68 parts by weight of epoxy resin jER-828 (produced by Japan Epoxy Resin Co., Ltd.), and the mixture was thoroughly mixed in a vacuum mixer ARV-310 (produced by THINKY Corporation). Then, 32 parts by weight of crosslinking agent, jER Cure ST-12 (produced by Japan Epoxy Resin Co., Ltd.) was mixed in the vacuum mixer ARV-310 to produce a compound. This compound was put into a metal mold of 100 mm×100 mm×30 mm and was heated under a pressure of 9.8 MPa (100 kg/cm²) at normal temperature for 4 hours and at 60° C. for 3 hours with a vacuum electric heat press machine to form a matching material block 2. This block was cut into a test piece of 50 mm×50 mm×2 mm for evaluation of the density and sound velocity. These were evaluated by the methods described below. The results were as follows: the density was 1.12 g/cm³, the sound velocity was 1750 m/s, and the acoustic impedance was 2.0 MRayls. The block was cut into 0.50 mm thick with a wire-saw CS-203 (produced by Musashino Denshi, Inc.) and was then ground into 0.050 mm thick with a precision grinding apparatus MA-200 (produced by Musashino Denshi, Inc.) to produce a matching material 2. As illustrated in Table 1 below, fillers, epoxy resins, and crosslinking agents were changed to produce matching materials 1, and 3 to 13. Other matching materials 14 to 16 were also prepared. The density, the sound velocity, and the acoustic impedance of each matching material are shown in Table 1 below.

### <Evaluation Procedure>

### (Density)

The density was measured with an electronic densitometer SD-200L (produced by Alfa Mirage Co., Ltd.) in accordance with Method A for measuring the density (by displacement of water) described in JIS-K7112 02.

### (Acoustic Characteristics)

The ultrasound velocity was measured at 25° C. in accordance with JIS Z2353-2003 with a sing-around sound velocity measuring apparatus produced by ULTRASOUND ENGINEERING Co., Ltd., and the acoustic impedance was calculated from the following equation:

Acoustic impedance (Z: MRayls)=Density (ρ: ×10³ kg/m³)×Sound velocity (C: ×10³ m/sec)

The ultrasound attenuation was determined in accordance with JIS Z2354-1992 by generating ultrasound waves of 1 MHz with an ultrasound pulser receiver JPR-10C (produced by Japan Probe Co., Ltd.) in a container filled with water at 25° C. and then by measuring the amplitudes of the ultrasound waves before and after the ultrasound waves penetrate through a sheet.

TABLE 1

| | | | | | SOUND | ACOUSTIC | |
|---|---|---|---|---|---|---|---|
| | | | | DENSITY | VELOCITY | IMPEDANCE | ATTENUATION |
| | EPOXY RESIN | CROSSLINKING AGENT | FILLER | g/cm³ | m/s | MRayls | dB/mm · MHz |
| 1 | JER-828 | JER CURE ST-12 | — | 1.16 | 2500 | 2.9 | 0.73 |
| | 68 | 32 | | | | | |
| 2 | JER-828 | JER CURE ST-12 | KMP600 | 1.12 | 1750 | 2.0 | 0.65 |
| | 68 | 32 | 25 | | | | |
| 3 | JER-828 | JER CURE ST-12 | KMP600 | 1.04 | 1450 | 1.5 | 0.56 |
| | 68 | 32 | 115 | | | | |
| 4 | JER-828 | JER CURE ST-12 | SX8782P | 1.02 | 2460 | 2.5 | 0.71 |
| | 68 | 32 | 200 | | | | |
| 5 | C1001A | C-1001B | — | 1.21 | 2500 | 3.0 | 0.54 |
| | 80 | 20 | | | | | |
| 6 | C1001A | C-1001B | FERRITE | 1.80 | 2350 | 4.2 | 0.60 |
| | 80 | 20 | 70 | | | | |
| 7 | C1001A | C-1001B | FERRITE | 2.60 | 2640 | 6.9 | 0.55 |
| | 80 | 20 | 340 | | | | |
| 8 | C1001A | C-1001B | FERRITE | 3.23 | 2850 | 9.2 | 0.45 |
| | 80 | 20 | 470 | | | | |
| 9 | C1001A | C-1001B | FERRITE | 3.94 | 3540 | 13.9 | 0.45 |
| | 80 | 20 | 1250 | | | | |
| 10 | EP007K MAIN AGENT | EP007K CURING AGENT | — | 1.10 | 2200 | 2.4 | 0.60 |
| | 50 | 50 | | | | | |
| 11 | EP007K MAIN AGENT | EP007K CURING AGENT | FERRITE | 1.80 | 2100 | 3.8 | 0.80 |
| | 50 | 50 | 105 | | | | |
| 12 | EP007K MAIN AGENT | EP007K CURING AGENT | FERRITE | 2.92 | 2420 | 7.1 | 0.70 |
| | 50 | 50 | 400 | | | | |
| 13 | EP007K MAIN AGENT | EP007K CURING AGENT | FERRITE | 3.92 | 2900 | 11.4 | 0.60 |
| | 50 | 50 | 1200 | | | | |

| | | | | | |
|---|---|---|---|---|---|
| 14 | POLYETHYLENE EL-N-AN | 0.96 | 2400 | 2.3 | 0.50 |
| | (MADE BY SHIN-KOBE ELECTRIC MACHINERY) | | | | |
| 15 | TPX MX002 (MADE BY MITSUI CHEMICALS) | 0.83 | 2200 | 1.7 | 0.45 |
| 16 | MACOR (MADE BY ISHIHARA CHEMICAL) | 2.52 | 5580 | 14.1 | 0.56 |

In Table 1, C1001A and C1001B were available from TESK Co., Ltd., and an EP007K main agent and an EP007K curing agent were available from Cemedine Co., Ltd.

### <Production and Evaluation of Acoustic Matching Layers>

Matching materials 2, 11, and 12 were laminated in this order. The laminate was pre-cured with an epoxy adhesive E set L (produced by Konishi Co., Ltd.) at normal temperature (25° C.) for 5 minutes under a pressure of 5 Kgf and then was subsequently bonded at normal temperature (25° C.) for 5 hours and then at 50° C. for 3 hours under a pressure of 30 Kgf to give an acoustic matching layer 1. Acoustic matching layers 2 to 10 were similarly produced as illustrated in Tables 3-1 and 3-2 below, the acoustic matching layers 1 to 10 were each tested for evaluation of the thickness of the film, the thickness and bonding strength of the adhesive layer, and the appearance. The physical properties of the adhesives used are shown in Table 2 below.

### (Appearance)

The surfaces of the matching materials were ground and were observed with a differential interference contrast microscope MX-50 (produced by Olympus Corporation) to check the cracks, and were evaluated based on the following five levels: A (no crack), B (cracks of equal to or less than 0.1 mm), C (cracks from 0.2 to 0.5 mm), D (cracks of equal to or more than 0.5 mm), and E (several cracks of equal to or more than 0.5 mm over the entire surface).

### (Layer Thickness)

The layer thickness was the average of six points measured with a contact type film thickness gauge K351-C (produced by ANRITSU Corporation).

### (Thickness of Adhesive Layer)

The cross-sectional surface of each matching material was pretreated with a LEICA normal temperature/frozen section producing ultramicrotome EM UC7i produced by Hitachi High-Technologies Corporation and was observed at an acceleration voltage of 200 kV at a 200-fold magnification with an electron microscope S-800 produced by Hitachi High-Technologies Corporation, and the thickness of the adhesive layer was measured.

### (Adhesiveness)

A 90-degree peel test in accordance with K6854-1:1999 (ISO 8510-1:1990) was performed at 25° C. and 50° C. with a digital force gauge ZP-20N (produced by IMADA Co., Ltd.) and a measurement stand MX-500N (produced by IMADA Co., Ltd.). Samples were produced similarly to the production of an ultrasound probe, and the peel strength of each sample was evaluated across a width of 1 cm.

TABLE 2

| | | | GLASS | |
|---|---|---|---|---|
| | | | TRANSITION | |
| | | VISCOSITY | TEMPERATURE | |
| | ADHESIVE | (25° C.) | (Tg) | |
| 1 | E SET L | 4.3 Pa · s | 35° C. | EXAMPLE |
| | (KONISHI) | | | |
| 2 | C1163 | 0.40 Pa · s | 55° C. | EXAMPLE |
| | (TESK) | | | |
| 3 | C1054 | 0.78 Pa · s | 60° C. | EXAMPLE |
| | (TESK) | | | |
| 4 | DP460 | 35 Pa · s | 73° C. | COMPARATIVE |
| | (SUMITOMO | | | EXAMPLE |
| | 3M) | | | |
| 5 | EP007 | 23 Pa · s | 52° C. | COMPARATIVE |
| | (CEMEDINE) | | | EXAMPLE |

TABLE 3-1

| | ACOUSTIC MATCHING LAYER | | | | |
|---|---|---|---|---|---|
| | FIRST LAYER | SECOND LAYER | THIRD LAYER | FOURTH LAYER | FIFTH LAYER |
| 1 | MATCHING | MATCHING | MATCHING | | |
| | MATERIAL-2 | MATERIAL-11 | MATERIAL-12 | | |
| | 0.04 mm | 0.04 mm | 0.05 mm | | |
| 2 | MATCHING | MATCHING | MATCHING | | |
| | MATERIAL-2 | MATERIAL-11 | MATERIAL-12 | | |
| | 0.04 mm | 0.04 mm | 0.05 mm | | |
| 3 | MATCHING | MATCHING | MATCHING | | |
| | MATERIAL-2 | MATERIAL-11 | MATERIAL-12 | | |
| | 0.04 mm | 0.04 mm | 0.05 mm | | |
| 4 | MATCHING | MATCHING | MATCHING | | |
| | MATERIAL-2 | MATERIAL-11 | MATERIAL-12 | | |
| | 0.04 mm | 0.04 mm | 0.05 mm | | |
| 5 | MATCHING | MATCHING | MATCHING | | |
| | MATERIAL-2 | MATERIAL-11 | MATERIAL-12 | | |
| | 0.04 mm | 0.04 mm | 0.05 mm | | |
| 6 | MATCHING | MATCHING | MATCHING | MATCHING | |
| | MATERIAL-4 | MATERIAL-6 | MATERIAL-7 | MATERIAL-13 | |
| | 0.04 mm | 0.04 mm | 0.04 mm | 0.05 mm | |
| 7 | MATCHING | MATCHING | MATCHING | MATCHING | MATCHING |
| | MATERIAL-15 | MATERIAL-10 | MATERIAL-6 | MATERIAL-12 | MATERIAL-13 |
| | 0.04 mm | 0.04 mm | 0.05 mm | 0.05 mm | 0.05 mm |
| 8 | MATCHING | MATCHING | MATCHING | MATCHING | MATCHING |
| | MATERIAL-2 | MATERIAL-5 | MATERIAL-7 | MATERIAL-8 | MATERIAL-16 |
| | 0.04 mm | 0.04 mm | 0.04 mm | 0.04 mm | 0.04 mm |
| 9 | MATCHING | MATCHING | MATCHING | | |
| | MATERIAL-2 | MATERIAL-11 | MATERIAL-12 | | |
| | 0.04 mm | 0.04 mm | 0.05 mm | | |
| 10 | MATCHING | MATCHING | MATCHING | | |
| | MATERIAL-2 | MATERIAL-11 | MATERIAL-12 | | |
| | 0.04 mm | 0.04 mm | 0.05 mm | | |

| | | CURING CONDITION | | | | |
|---|---|---|---|---|---|---|
| | | TEMPERATURE/ | | | | |
| | | TIME | | | | |
| | | INITIAL | | PRESSURE/TIME | | |
| | ADHESIVE | CONDITION | CONDITION | PRE-CURING | CONDITION | |
| 1 | E SET L | 25° C. | 50° C. | 5 kgf | 30 kgf | EXAMPLE |
| | | 5 HOURS | 3 HOURS | 5 MINUTES | 8 HOURS | |
| 2 | C1163 | 25° C. | 50° C. | 5 kgf | 30 kgf | EXAMPLE |
| | | 5 HOURS | 3 HOURS | 5 MINUTES | 8 HOURS | |
| 3 | E SET L | 50° C. | — | 5 kgf | 30 kgf | EXAMPLE |
| | | 4 HOURS | | 5 MINUTES | 8 HOURS | |
| 4 | E SET L | 50° C. | — | — | 30 kgf | EXAMPLE |
| | | 4 HOURS | | | 4 HOURS | |
| 5 | C1054 | 50° C. | — | — | 30 kgf | EXAMPLE |
| | | 4 HOURS | | | 4 HOURS | |
| 6 | C1163 | 25° C. | 50° C. | 5 kgf | 30 kgf | EXAMPLE |
| | | 5 HOURS | 3 HOURS | 5 MINUTES | 8 HOURS | |
| 7 | C1163 | 25° C. | 50° C. | 5 kgf | 30 kgf | EXAMPLE |
| | | 5 HOURS | 3 HOURS | 5 MINUTES | 8 HOURS | |
| 8 | C1163 | 25° C. | 50° C. | 5 kgf | 30 kgf | EXAMPLE |
| | | 5 HOURS | 3 HOURS | 5 MINUTES | 8 HOURS | |
| 9 | DP460 | 50° C. | — | — | 30 kgf | COMPARATIVE |
| | | 4 HOURS | | | 4 HOURS | EXAMPLE |
| 10 | EP007 | 50° C. | — | — | 30 kgf | COMPARATIVE |
| | | 4 HOURS | | | 4 HOURS | EXAMPLE |

TABLE 3-2

| | ACOUSTIC MATCHING LAYER | | | | |
|---|---|---|---|---|---|
| | FIRST LAYER | SECOND LAYER | THIRD LAYER | FOURTH LAYER | FIFTH LAYER |
| 1 | MATCHING | MATCHING | MATCHING | | |
| | MATERIAL-2 | MATERIAL-11 | MATERIAL-12 | | |
| | 0.04 mm | 0.04 mm | 0.05 mm | | |
| 2 | MATCHING | MATCHING | MATCHING | | |
| | MATERIAL-2 | MATERIAL-11 | MATERIAL-12 | | |
| | 0.04 mm | 0.04 mm | 0.05 mm | | |
| 3 | MATCHING | MATCHING | MATCHING | | |
| | MATERIAL-2 | MATERIAL-11 | MATERIAL-12 | | |
| | 0.04 mm | 0.04 mm | 0.05 mm | | |
| 4 | MATCHING | MATCHING | MATCHING | | |
| | MATERIAL-2 | MATERIAL-11 | MATERIAL-12 | | |
| | 0.04 mm | 0.04 mm | 0.05 mm | | |
| 5 | MATCHING | MATCHING | MATCHING | | |
| | MATERIAL-2 | MATERIAL-11 | MATERIAL-12 | | |
| | 0.04 mm | 0.04 mm | 0.05 mm | | |
| 6 | MATCHING | MATCHING | MATCHING | MATCHING | |
| | MATERIAL-4 | MATERIAL-6 | MATERIAL-7 | MATERIAL-13 | |
| | 0.04 mm | 0.04 mm | 0.04 mm | 0.05 mm | |
| 7 | MATCHING | MATCHING | MATCHING | MATCHING | MATCHING |
| | MATERIAL-15 | MATERIAL-10 | MATERIAL-6 | MATERIAL-12 | MATERIAL-13 |
| | 0.04 mm | 0.04 mm | 0.05 mm | 0.05 mm | 0.05 mm |
| 8 | MATCHING | MATCHING | MATCHING | MATCHING | MATCHING |
| | MATERIAL-2 | MATERIAL-5 | MATERIAL-7 | MATERIAL-8 | MATERIAL-16 |
| | 0.04 mm | 0.04 mm | 0.04 mm | 0.04 mm | 0.04 mm |
| 9 | MATCHING | MATCHING | MATCHING | | |
| | MATERIAL-2 | MATERIAL-11 | MATERIAL-12 | | |
| | 0.04 mm | 0.04 mm | 0.05 mm | | |
| 10 | MATCHING | MATCHING | MATCHING | | |
| | MATERIAL-2 | MATERIAL-11 | MATERIAL-12 | | |
| | 0.04 mm | 0.04 mm | 0.05 mm | | |

| | | | THICKNESS | | | |
|---|---|---|---|---|---|---|
| | | | OF | | | |
| | | THICKNESS | ADHESIVE | PEEL STRENGTH | | |
| | APPEARANCE | OF LAYER | LAYER | 25° C. | 50° C. | |
| 1 | A | 0.127 mm | 0.3 μm | 0.56 kgf/cm | 0.11 kgf/cm | EXAMPLE |
| 2 | A | 0.127 mm | 0.3 μm | 0.53 kgf/cm | 0.49 kgf/cm | EXAMPLE |
| 3 | A | 0.127 mm | 0.6 μm | 0.55 kgf/cm | 0.11 kgf/cm | EXAMPLE |
| 4 | A | 0.127 mm | 0.8 μm | 0.57 kgf/cm | 0.09 kgf/cm | EXAMPLE |
| 5 | A | 0.127 mm | 0.7 μm | 0.54 kgf/cm | 0.49 kgf/cm | EXAMPLE |
| 6 | A | 0.165 mm | 0.2 μm | 0.54 kgf/cm | 0.50 kgf/cm | EXAMPLE |
| 7 | A | 0.221 mm | 0.3 μm | 0.53 kgf/cm | 0.50 kgf/cm | EXAMPLE |
| 8 | A | 0.196 mm | 0.2 μm | 0.52 kgf/cm | 0.41 kgf/cm | EXAMPLE |
| 9 | E | 0.125 mm | 1.4 μm | 0.53 kgf/cm | 0.49 kgf/cm | COMPARATIVE |
| | | | | | | EXAMPLE |
| 10 | E | 0.125 mm | 1.2 μm | 0.53 kgf/cm | 0.25 kgf/cm | COMPARATIVE |
| | | | | | | EXAMPLE |

It was found that the acoustic matching layers, which were produced by bonding the matching materials with adhesives having low viscosity and high glass transition temperatures (Tg) in this way, caused no crack and had high bonding strengths and high yield rates.

The present examples performs pre-curing involving press under a low pressure of 5 Kgf before bonding under a high pressure of 30 Kgf as explained above. This can further reduce the damage of the matching material due to the shear stress.

### <Production of Backing Layer>

To 91 parts by weight of a liquid silicone rubber TSE3032 (A) (produced by Momentive Performance Materials Inc.) was added 750 parts by weight of tungsten trioxide powder A₂-WO₃ (produced by Allied Material Co., Ltd.), and the mixture was thoroughly mixed in a vacuum mixer ARV-310 (produced by THINKY Corporation). Then, 9 parts by weight of TSE3032 (B) was added and mixed sufficiently. This mixture was put into a metal mold of 100 mm×100 mm×30 mm and was heated under a pressure of 4.9 MPa (50 kg/cm²) at normal temperature under vacuum for 3 hours and at 50° C. for 3 hours with a vacuum electric heat press machine to produce a block of compound particles. This block had a density of 7.3 g/cm³. This block was cut into 1 cm square, was roughly smashed with a cutter mill VM-20 (produced by Makino mfg Co., Ltd.) and then smashed primarily with a screen of 0.5 mm at a revolution of 2800 rpm with a pin mill M-4 (produced by Nara machinery Co., Ltd.). The resultant particles were then sifted through an opening of 212 μm with a circular vibration sifting machine KG-400 (produced by Nishimura Machine Works Co., Ltd.) into filler compound particles.

The average particle diameter was 123 μm that was measured with a laser type particle size distribution measuring machine LMS-30 (produced by Seishin Enterprise Co., Ltd.).

To 91 parts by weight of an epoxy resin Albidur EP2240 (produced by NANORESINS Co.) was added 380 parts by weight of the filler compound particles, and the mixture was thoroughly mixed in a vacuum mixer ARV-310 (produced by THINKY Corporation). Then, 9 parts by weight of a crosslinking agent, jER Cure ST-12 (produced by Japan Epoxy Resin Co., Ltd.) was mixed in the vacuum mixer ARV-310 to produce a compound. This compound was put into a metal mold of 100 mm×100 mm×30 mm and was heated under a pressure of 9.9 MPa (100 kg/cm²) at normal temperature for 4 hours and at 60° C. for 3 hours with a vacuum electric heat press machine to form a backing block. This block had a density of 2.65 g/cm³, an acoustic impedance of 2.9 MRayls, and an attenuation of 30 dB/cm·MHz. This block was cut into 6 mm thick with a wire-saw CS-203 (produced by Musashino Denshi, Inc.) and was then ground into 5 mm thick with a precision grinding apparatus MA-200 (produced by Musashino Denshi, Inc.) to produce a backing layer.

### <Production of Acoustic Lens>

Fine particle zinc oxide Zincox Super F-2 (produced by HakusuiTech Co., Ltd.) was thinly put onto a stainless steel tray which was then put into a drier at 250° C. and was dried for 4 hours to eliminate surface-adsorbed water. At this time, the mass decreased by 0.7% by mass. Then, 40 parts by weight of the fine particles were kneaded with 100 parts by weight of a silicone rubber compound KE742U (produced by Shin-Etsu Silicon) with a roll kneading machine No. 191-TM/WM test mixing roll (produced by Yasuda Seiki Seisakusho, Ltd.) to prepare a rubber composition. Subsequently, 100 parts by weight of this rubber composition was roll-mixed with 0.5 parts by weight of 2,5-dimethyl-2,5-di(t-butyl peroxy)hexane as a vulcanizing agent to prepare a compound for molding. This compound for molding was press-molded at 165° C. for 10 minutes with a P500F-4141 manual molding machine (produced by Shoji Co., Ltd.) and then secondarily vulcanized at 200° C. for 2 hours to produce an acoustic lens. This acoustic lens had an acoustic impedance of 1.3 MRayls and an acoustic attenuation of −0.7 dB/mm·MHz.

### <Production of Ultrasound Probe>

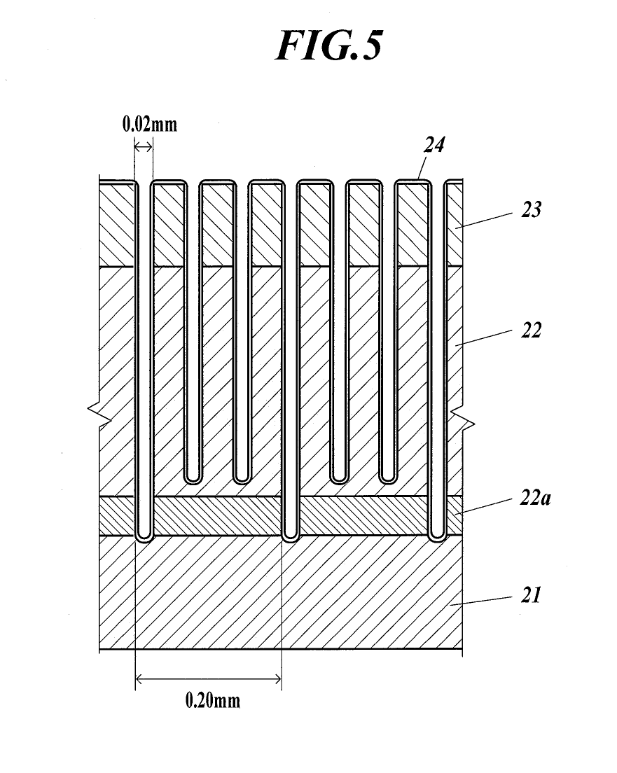

The backing layer, the FPC, the piezoelectric layer, and the acoustic matching layer were laminated in this order. The piezoelectric layer was composed of a piezoelectric material PZT 3203HD (produced by CTS Electro Component Inc.) having a thickness of 0.13 mm, and the acoustic matching layer was composed of the acoustic matching layer 9. The lamination of these layers was diced at a pitch of 0.20 mm with a dicer having a thickness of 0.02 mm into individual elements as illustrated in FIG. 5. Each element diced at a pitch of 0.20 mm was further diced into three equal fragments so as not to cut the electrode of the element.

The element was then coated with a polychloroparaxylylene film by putting dix-C (produced by Kisco Ltd.) as a raw material dimer into LABCOTER PDS2010 such that the film had a thickness of 3 μm. At this time, a single film of polychloroparaxylylene was produced to obtain its acoustic impedance equal to 2.8 MRayls from its density and acoustic characteristics.

Dice slots formed by the dicing were filled with an RTV silicon adhesive KE-1600 (produced by Shin-Etsu Silicon) under vacuum. The acoustic lens described above was then pressed and bonded with the RTV silicon adhesive KE-1600 under vacuum to produce an ultrasound probe 1 (Comparative Example 1). As illustrated in Table 4 below, ultrasound probes 2 and 3, respectively, were produced as Examples 1 and 2 similarly. Similarly to the ultrasound probe described above, the heavy backing layer (WC/Co plate) was sandwiched between the FPC and the piezoelectric layer into an ultrasound probe 4 (Example 3) as illustrated in Table 4. In this example, the piezoelectric material used was composed of PZT C6 (produced by Fuji Ceramics Corporation) having a thickness of 0.09 mm. The WC/Co plate was prepared by cutting and grinding FRT15 (produced by SANALLOY INDUSTRY Co., Ltd.) into a thickness illustrated in Table 4 below. The surface roughness Ra was 0.10 um that was measured with a laser confocal microscope LEXT OLS4000.

### <Evaluation of Acoustic Characteristics>

The ultrasound probes produced as described above were evaluated for the acoustic characteristics with FirstCall 2000 (produced by Sonora Medical Systems, Inc). The sensitivity, band characteristics, and deficiency rate of the elements are illustrated in Table 4 below.

TABLE 4

| | | PIEZO- | ACOUSTIC | | | | | |
|---|---|---|---|---|---|---|---|---|
| ULTRASOUND | HEAVY | ELECTRIC | MATCHING | RELATIVE | BAND | | DEFICIENCY | |
| PROBE | BACKING | MATERIAL | LAYER | SENSITIVITY | BW6 | BW20 | RATE | |
| 1 | — | 3203HD | ACOUSTIC | 100 | 77% | 123% | 2.6% | COMPARATIVE |
| | | 0.15 mm | MATCHING | | | | | EXAMPLE 1 |
| | | | LAYER-9 | | | | | |
| 2 | | 3203HD | ACOUSTIC | 102 | 83% | 130% | 0.0% | EXAMPLE 1 |
| | | 0.15 mm | MATCHING | | | | | |
| | | | LAYER-7 | | | | | |
| 3 | | 3203HD | ACOUSTIC | 102 | 79% | 132% | 0.0% | EXAMPLE 2 |
| | | 0.15 mm | MATCHING | | | | | |
| | | | LAYER-6 | | | | | |
| 4 | WC/Co | C6 | ACOUSTIC | 121 | 77% | 129% | 0.0% | EXAMPLE 3 |
| | 0.06 mm | 0.09 mm | MATCHING | | | | | |
| | | | LAYER-6 | | | | | |

It was found that the acoustic matching layers, which were produced by bonding the matching materials with adhesives having low viscosity, had small thicknesses of the adhesive layers, low deficiency rates, and high yield rates.

According to the embodiment as explained above, at least one of two or more of the matching materials 23a to 23c includes a thermosetting resin and a filler for adjusting the acoustic impedance while two or more matching materials 23a to 23c are bonded with an adhesive having viscosity of 10 Pa·s or less at 25° C. This process can prevent the matching material from receiving damages, such as cracks, due to the shear stress during press of the matching material in the bonding process of the matching material to manufacture an ultrasound probe having a high yield rate.

Moreover, the embodiment involves an adhesive having a glass transition temperature of 50° C. or more after curing and can therefore prevent separation of the matching materials due to deterioration of the adhesive caused by heat generated during the manufacture of elements by dicing.

The embodiment also involves the foremost matching material 23c of the acoustic matching layer 23 containing silicone resin particles and can therefore decrease the acoustic impedance more effectively.

According to the embodiment, the bonding process of two or more of the matching materials 23a to 23c with the adhesive includes a first step of holding the two or more of the matching materials 23a to 23c at a predetermined temperature for a predetermined period until the thickness of the adhesive layer reaches a predetermined thickness from the start of the bonding, and a second step of subsequently holding the two or more of the matching materials 23a to 23c at higher temperature. These steps enable the adhesive layer to have a smaller thickness than a predetermined value and can effectively enhance the adhesive strength. These steps can also provide a wide applicable range of the adhesive.

According to the embodiment, the two or more of the matching materials 23a to 23c are also held at 15° C. or more and 30° C. or less for 2 to 6 hours in the first step and 60° C. or less for 1 to 4 hours in the second step. These steps can provide a more appropriate thickness of the adhesive layer and effectively enhance the adhesive strength.

The description on the embodiments of the present invention is an example of an ultrasound diagnostic imaging apparatus according to the present invention; however, the present invention is not limited to this. Detailed configurations and operations of the functional units in the ultrasound diagnostic imaging apparatus can also be properly modified.

The entire disclosure of Japanese Patent Application No. 2013-040215 filed on Mar. 1, 2013 including description, claims, drawings, and abstract are incorporated herein by reference in its entirety.

What is claimed is:

1. A method for manufacturing an ultrasound probe which includes a piezoelectric element for transmitting and receiving ultrasound waves and an acoustic matching layer provided by lamination of two or more matching materials on a front surface of the piezoelectric element, the method comprising:
forming at least one of the two or more matching materials from a thermosetting resin and a filler for adjusting an acoustic impedance; and
bonding the two or more matching materials to each other with an adhesive having a viscosity of 10 Pa·s or less at 25° C.

2. The method for manufacturing the ultrasound probe according to claim 1, wherein the adhesive has a glass transition temperature of 50° C. or more after curing.

3. The method for manufacturing the ultrasound probe according to claim 1, wherein a matching material which is a foremost layer in the acoustic matching layer contains silicone resin particles.

4. The method for manufacturing the ultrasound probe according to claim 1, wherein the bonding of the two or more matching materials with the adhesive includes a first step of holding the two or more matching materials at a predetermined temperature for a predetermined period until a thickness of an adhesive layer becomes a predetermined thickness or less from a start of the bonding and a second step of subsequently holding the two or more matching materials at a higher temperature.

5. The method for manufacturing the ultrasound probe according to claim 4, wherein the two or more matching materials are held at 15° C. or more and 30° C. or less for 2 to 6 hours in the first step and at 60° C. or less for 1 to 4 hours in the second step.

6. An ultrasound diagnostic imaging apparatus comprising:
an ultrasound probe manufactured by the method for manufacturing the ultrasound probe according to claim 1, the ultrasound probe outputting transmission ultrasound toward a subject in response to a driving signal and receiving reflected ultrasound from the subject to output a received signal; and
an image processing unit for generating ultrasound image data for displaying an ultrasound image based on the received signal outputted from the ultrasound probe.
